(19) United States
(12) Patent Application Publication
(10) Pub. No.: US 2010/0021409 A1
(43) Pub. Date: Jan. 28, 2010

(54) Film Forming Personal Care Compositions

(76) Inventor: Ali Abdelaziz Alwattari, West Chester, OH (US)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/408,664

(22) Filed: Mar. 20, 2009

Related U.S. Application Data
(63) Continuation-in-part of application No. 12/368,910, filed on Feb. 10, 2009.
(60) Provisional application No. 61/027,544, filed on Feb. 11, 2008.

(51) Int. Cl. A61K 8/72; A61K 8/73; A61K 8/81; A61K 8/00; A61Q 9/02

(52) U.S. Cl. 424/70.13; 424/70.11; 424/70.15; 424/70.17; 424/73

(57) ABSTRACT

A shaving composition, comprising a base composition, comprising a film forming system comprising one or more film forming materials; and one or more water soluble or water dispersible surface active agents; and at least one suspended benefit agent disposed within the base composition.

### CROSS REFERENCE TO RELATED APPLICATION

This application claims the benefit of U.S. application Ser. No. 12/368,910, filed Feb. 10, 2009, which claims the benefit of U.S. Provisional Application Ser. No. 61/027,544, filed Feb. 11, 2008.

### FIELD OF THE INVENTION

The present invention relates to personal care compositions comprising a film forming system. The personal care compositions provided herein are particularly suitable as shaving compositions. Note however that although the background and detailed description sections are substantially focused on shaving-related compositions, the personal care compositions of the present invention may be used/marketed for different applications within the area of personal care (including for both human and domesticated animals).

The present invention also relates to shaving products, shaving kits, shaving systems, and methods of shaving.

### BACKGROUND OF THE INVENTION

Many consumers of shave preparations prefer lathering products, and for this reason, the art remains focused on surfactant or soap based shave formulations that deliver a rich lather. However, the use of lathering compositions has generally represented a trade-off in which protection is sacrificed for aesthetics. This trade-off is the result of a conflict between surfactants which can provide pleasing aesthetics and functional components which can provide improved protection and/or other shaving or post-shave skin benefits.

One recognized and particularly beneficial class of such functional components is film forming polymers. A non-exclusive list of aqueous based film forming polymers is disclosed in U.S. Application Publication No. 2006/0228319 (Vona et al.). Film forming polymers are known to provide numerous benefits; for example, in U.S. Pat. No. 5,500,211 (George et al.), film forming polymers are said to provide improved lubricity and post-shave skin feel.

Recognition of the benefits provided by film forming polymers has not, however, resulted in a change in the shave preparation formulation paradigm. The failure of film forming polymers to revolutionize shave compositions is due to challenges to formulate high levels of film forming polymers into consumer acceptable shave preparations. This inability is the result of polymer-surfactant interaction which can lead to unacceptable rheologies.

Numerous attempts have been made to gain consumer acceptance for nontraditional product forms which facilitate the inclusion of high levels of film forming polymers; for example, the dry films disclosed in U.S. Application Publication No. 2006/0228319 (Vona et al.), or the viscous gels in U.S. Pat. No. 4,994,265 (White). However, these product forms have not gained wide acceptance, and many consumers remain focused on more traditional lathering product forms.

Traditional lathering product forms are heavily focused on surfactants and/or soaps. Surfactants provide a number of functional and aesthetic benefits including the rich lather which many consumers prefer. However, surfactants can also remove lipids from the stratum corneum which can damage the barrier function of the skin and can lead to skin dryness and irritation. The drying caused by high levels of surfactants can be mitigated through inclusion of humectants like glycerin, but these materials can contribute to an undesirable oily aesthetic. Moreover, surfactants are generally highly water dispersible, and therefore offer poor sustained protection throughout the shaving experience. Under the aqueous conditions of typical shaving practices, surfactant-based shave compositions can quickly wash away leaving the skin substantially unprotected when multiple razor strokes are directed to a region of skin.

### DETAILED DESCRIPTION OF ILLUSTRATIVE AND PREFERRED EMBODIMENTS OF THE INVENTION

The present invention may be understood more readily by reference to the following detailed description of illustrative and preferred embodiments. It is to be understood that the scope of the claims is not limited to the specific ingredients, methods, conditions, devices, or parameters described herein, and that the terminology used herein is not intended to be limiting of the claimed invention. Also, as used in the specification, including the appended claims, the singular forms “a,” “an,” and “the” include the plural, and reference to a particular numerical value includes at least that particular value, unless the context clearly dictates otherwise. When a range of values is expressed, another embodiment includes from the one particular value and/or to the other particular value. Similarly, when values are expressed as approximations, by use of the antecedent basis “about,” it will be understood that the particular values form another embodiment. All ranges are inclusive and combinable.

All percentages and ratios used herein are by weight of the total composition, and all measurements made are at 25° C., unless otherwise designated.

The embodiments of the present invention can comprise, consist of, and consist essentially of the features and/or steps of the invention described herein, as well as any of the additional or optional ingredients, components, steps, or limitations described herein or would otherwise be appreciated by the skilled artisan.

### I. Personal Care Compositions

The present invention is directed to personal care compositions that include a base composition and an optional lathering agent mixed with the base composition. The base compositions comprise a film forming system and one or more water soluble or water dispersible surface active agents. These components and additional optional components (e.g., thickeners/structurants and humectants) will be discussed more fully below. Exemplary base compositions include from about 20% to about 80% water. It should be noted that one embodiment provided by the present invention is in a “dried down” product form (e.g., a foamed film, pad, or pellets), wherein the base composition originally contained a significant level of water prior to being manipulated into its solid form.

In one embodiment, personal care compositions of the present invention have a viscosity of from about 150 to about 3000 cps, in an alternate embodiment from about 185 to about 2500 cps, in an alternate embodiment from about 190 to about 2000 cps, in an alternate embodiment from about 200 to about 1900 cps as measured by a commercial dip probe rheometer, such as the Hydramotion Viscolite 700 Model VL700s (solid state insertion viscosity meter). The viscosity in the current invention is determined by submerging the testing probe into 200 milliliters of the formulation, activate the probe, and record the result after 60 seconds of stabilization time.

Without wishing to be bound by theory, it is believed that personal care compositions of the present invention having increased viscosity provide for superior protection because they are more effective at high shear rates. Moreover, the high viscosities of the present invention surprisingly occur while maintaining substantially the same coefficient of friction as traditional shaving compositions, providing superior protection for the skin.

A. Film Forming System

Personal care compositions of the present invention contain a film forming system. The film forming system can be made up of at least one film forming material. In certain embodiments, it may be beneficial for more than one film forming material to make up the film forming system.

Useful film forming materials include, but are not limited to, polyvinylpyrrolidone, polyethylene oxide, hydroxyethylcellulose, hydroxylpropylcellulose, starch, polyvinyl alcohol, albumins, cationic celluloses, xanthan, carageenan, sodium polystyrene sulfonate, sodium silicone t-butyl acrylate, sodium poly (styrene sulfonate/maleic anhydride), sodium poly (styrene sulfonate co acrylate), polyvinylsulfonate, polyvinyl sulfate, polyphosphate, polymethacrylate, sodium dextran sulphate, poly (ethylene oxide co styrene sulfonate), methylcellulose, hydroxypropylmethylcellulose, ethylhydroxyethylcellulose, methylhydroxyethylcellulose, agar, dextran, amphomer, celquat, glucamate DOE-120, Glucamate LT, polyquaterniums (e.g., PQ 2, 7, 10, 16, 17, 18, 19, 24, 27, and 46), Merquats, Quaternized PVP, proteins and polypeptides (e.g., collagen, elastin, keratin, and their quaternary derivatives such as CROQUAT and QUAT-Coll), adipic acid/dimethylaminohydroxypropyl/diethylenetriamine copolymer), PVP/methacrylate, Aquaflex (polyimide-1), Gantrez (copolymers of methyl vinyl ether and maleic anhydride) Styleze (vinyl pyrrolidone/acrylate/lauryl methacrylate copolymer), pectin, and mixtures thereof.

Other suitable film forming materials include acrylic copolymer emulsion, silicone elastomers latex, polyurethane latex, styrene-acrylate latex, styrene-butadiene latex, swollen hydrogels, polyvinylacrylate, polyacrylamide, Eastman AQ polyester-thiosulphate polymers, polysilicates, self crosslinking polymer emulsions (e.g., those containing zinc or calcium that expedites and elasticizes the coalescing polymer latex spheres together), core-shell latexes, vinyl versatate copolymers, vinyl-acetate-ethylene copolymers, vinyl maleate copolymers, natural latex, copolymer of vinylidene chloride/acrylonitrile/methylmethacrylate, and mixtures thereof.

Additional film forming materials useful for employment in embodiments of the present invention include silicone-acrylate copolymers, acrylate/ethylhexyl acrylate/dimethicone methacrylate copolymer, acrylate/behenyl acrylate/dimethicone methacrylate copolymer, polyvinylbutyral, polyurethane, PVP/VA copolymer, styrene-ethylen/propylene-styrene elastomers, styrene-ethylene/butylene-styrene elastomers, terminal hydroxylated polyethylene/butylene elastomers, Kraton (styrene-isoprene copolymer), siloxysilicate copolymer, polydimethylsiloxane gums and resins, Ganex PVP/olefin polymers, ethylene-propylene elastomers, random thermoplastic elastomers, polystyrene-co-polyethylenepropylene elastomers, dermacryl, polysilsesquioxanes and other non-thickening substantivity agents known as dendrimers, polysiloxanes, silicone quaternium compounds, aminosilicones, silicone protein copolymers (e.g., CRODASONE W, and PECOSIL), nitroceullose, cyanoacrylates, and mixtures thereof.

In one exemplary shaving composition embodiment, the film forming system is defined by the combination of a lubricant polymer and a substantivity polymer. The lubricant polymer may include, for example, polyvinylpyrrolidone, polyethylene oxide, polyvinylpyrrolidone/vinyl alcohol copolymer, vinyl alcohol, vinyl acetate polymers and copolymers, and polyacrylamides. In certain embodiments of the present invention mixtures of different lubricant polymers are suitable; in others a single lubricant polymer is suitable. One class of useful lubricant polymers includes, but is not limited to, film forming polymers which have a molecular weight of greater than about 150,000 g/mol. There is theoretically no upper limit to the molecular weights of polymers which may constitute lubricant polymers, but practically, the limit is imposed by the molecular weight beyond which the polymer is no longer soluble/dispersible in the personal care composition. In certain applications, it is preferred that the lubricant polymer be substantially unbranched film forming polymers. The substantivity polymer may include, for example, cationic polymers, proteins, polypeptides, polysilsesquioxanes, and dendrimers. Non-limiting examples of aqueous-based film forming cationic polymers can be found in U.S. Pat. No. 4,240,450 (Grollier et al.) which is hereby incorporated in relevant part by reference. Some preferred substantivity polymers include polyquaternium-10 and polyquaternium-11. In certain applications, mixtures of two or more substantivity polymers are suitable; in others, employment of a single substantivity polymer is suitable.

Applicant believes, without wishing to be bound by theory, that the substantivity polymer, in a film forming system comprising a combination of a lubricant polymer and a substantivity polymer, forms an entangled mesh that can act to entrap the lubricant polymer. Since the substantivity polymer is selectively maintained under aqueous shave conditions, the lubricant polymer is also retained. This type of film forming system accordingly acts to provide sustained protection/lubrication. In exemplary sustained protection shaving embodiments, the lubricant polymer comprises from about 0.1% to about 2%, by weight of the shave composition, and the substantivity polymer comprises from about 0.1% to about 2%, by weight of the shave composition. In one preferred sustained protection embodiment, the lubricant polymer comprises from about 0.25% to about 1%, and the substantivity agent comprises from about 0.25% to about 1%. In another preferred sustained protection embodiment, the lubricant polymer is present in ranges from about 0.25% to about 0.6%, and the substantivity polymer is present in the range of about 0.5% to about 0.8%.

B. Water Soluble or Water Dispersible Surface Active Agent

Personal care compositions of the present invention contain one or more surface active agents. The water dispersible or water soluble surface active agent is preferably one that is capable of forming lather and may comprise a soap, an interrupted soap, a detergent, an anionic surfactant, a non-ionic surfactant or a mixture of one or more of these.

Soaps may include, for example, the sodium, potassium and lower alkanolamine (preferably triethanolamine) salts of C12 22, preferably C14 18, fatty acids. Typical fatty acids include lauric, myristic, palmitic and stearic acid and mixtures thereof. The preferred fatty acids are palmitic and stearic. The interrupted soaps may include, for example, the sodium, potassium and lower alkanolamine (preferably triethanolamine) salts of N-fatty acyl sarcosines, wherein the fatty acyl moiety has 12 to 22, preferably 14 to 18, carbon atoms. Typical sarcosines include stearoyl sarcosine, myristoyl sarcosine, palmitoyl sarcosine, oleoyl sarcosine, lauroyl sarcosine, cocoyl sarcosine and mixtures thereof. The soaps and the interrupted soaps may be utilized in the preneutralized form (i.e., as the sodium, potassium or alkanolamine salt) or in the free acid form followed by subsequent neutralization with sodium hydroxide, potassium hydroxide and/or lower alkanolamine (preferably triethanolamine). In any event, the final composition preferably contains sufficient base to neutralize or partially neutralize the soap component and adjust the pH to the desired level (typically between 5 and 10, more typically between 6 and 9). It is most preferred that the shaving composition includes a soap (e.g., triethanolamine palmitate/stearate), an interrupted soap (e.g., triethanolamine stearoyl/myristoyl sarcosinate), or a mixture thereof.

The water dispersible surface active agent may also optionally include a non-ionic, amphoteric and/or anionic surfactant. Suitable non-ionic surfactants will typically have an HLB of 9 or more and include the polyoxyethylene ethers of fatty alcohols, acids and amides, particularly those having 10 to 20, preferably 12 to 18, carbon atoms in the fatty moiety and about 2 to 60, preferably 4 to 30, ethylene oxide units. These include, for example, Oleth 20, Steareth 21, Ceteth 20, Laureth 4 and Laureth 23. Other non-ionic surfactants include the polyoxyethylene ethers of alkyl substituted phenols, such as Nonoxynol-4 and Nonoxynol-20, fatty alkanolamides such as Lauramide DEA and Cocamide MEA, polyethoxylated sorbitan esters of fatty acids, such as Polysorbate 20, lauryl polyglucoside, sucrose laurate, and polyglycerol 8 oleate. Suitable amphoteric surfactants include, for example, the betaines and sultaines such as cocoamidopropyl betaine, coco dimethyl carboxymethyl betaine, lauroamphoacetate, cocaminopropionic acid, and mixtures thereof. Others include isononyl isononanoate, polyhydroxystearic acid, ethylhexyl isononanoate, sodium cocamidopropyl PG-Dimonium chloride, Cetearyl alcohol, cholesterol, and stearyl alcohol. Suitable anionic surfactants include, for example, the sodium, potassium, ammonium and substituted ammonium salts (such as the mono-, di- and triethanolamine salts) of C8 C22, preferably C12 C18, alkyl sulfates (e.g., sodium lauryl sulfate, ammonium lauryl sulfate), alkyl sulfonates (e.g., ammonium lauryl sulfonate), alkylbenzene sulfonates (e.g., ammonium xylene sulfonate), acyl isethionates (e.g., sodium cocoyl isethionate), acyl lactylates (e.g., sodium cocoyl lactylate), alkyl ether sulfates (e.g., ammonium laureth sulfate, ammonium laurylether sulfate), sodium methyl cocoyl taurate, sodium lauryl sulfoacetate, and dioctyl sodium sulfosuccinate.

C. Concentration Levels of the Film Forming System & the Water Soluble or Water Dispersible Surface Active Agents

Applicant has discovered that shaving compositions that employ a certain minimum level of film forming materials and a particular film forming system to surface active agent ratio can provide a number of important shave-related benefits. In accordance with some embodiments, the minimum level of film forming materials is about 0.6%, by weight of the base composition, and the film forming system to surface active agent ratio is from about 0.03 to about 0.7 (note that the film forming system and surface active agent components defining this ratio may contain a single material or multiple materials). In accordance with other embodiments, the minimum level of film forming materials is about 0.6%, by weight of the base composition, and the film forming system to surface active agent ratio is from about 0.03 to about 0.2.

Sustained lubrication (or “controlled friction” from start to finish of the shaving process) is one of the shave-related benefits that can be provided by embodiments of the present invention. Typical surfactant-based shaving compositions that do not employ significant levels of film forming materials are generally highly water dispersible, and therefore offer poor sustained protection. Under the aqueous conditions of shaving, these shaving compositions quickly wash away leaving the skin minimal protection when multiple razor strokes are directed to a region of skin. This is a particularly unfortunate feature of shaving compositions directed toward the male consumer because facial hair is known to grow in an irregular manner. A closer shave can therefore be obtained by directing multiple strokes at the same region of skin but from different origination points. Shaving embodiments that employ greater than or equal to about 0.6 wt % of a film forming system and surface active agents at concentrations that define a film forming system to surface active agent ratio of from about 0.03 to about 0.7 are believed capable of providing sustained lubrication.

Controllable post-shave skin feel (adjustable to desired consumer segment: smooth, soft, “squeaky clean,” “draggy,” etc.) via the rinsing and frictional properties of the film forming system is another benefit not easily achievable with current technologies in the marketplace that may be provided by embodiments of the present invention. Many known shaving compositions can leave one's skin feeling dry or tight after shaving. The film forming system employed by embodiments of the present invention can include substantivity polymers that have an affinity for skin and have a reduced tendency to be completely washed or scraped off during a shaving routine. Film forming materials that remain on the skin after shaving can impart an improved post-shave skin feel. The choice and concentration level of film forming materials can be manipulated to target a more or less noticeable post-shave skin feel benefit. For example, employing 1.0 wt % or more of a film forming system may lead to a more positive post-shave skin feel. Increasing the film forming system to surface active agent ratio can also help provide a positive post-shave skin feel since significant amounts of surface active agents can tend to dry the skin.

Certain embodiments of the present invention are capable of providing increased cushion. Surfactant base bubbles (defining the “lather” or “foam” associated with shaving compositions) are made up of a gas surrounded by a skin of surfactant. These bubbles can withstand only a minimal force before rupturing. It is believed that the film forming materials of the present invention can act as a second skin for these bubbles. This second skin is believed to increase the force each bubble can withstand prior to rupture. The increased strength of each bubble is believed to increase the normal force that the shave composition exerts against an applied razor blade. The effect of increased shave composition cushion is a decrease in consumer awareness of the razor blade.

Additional exemplary shaving composition embodiments provided by the present invention include the following concentration levels of film forming materials and surface active agents: from about 0.6% wt % to about 1.2 wt % of film forming materials and from about 20 wt % to about 30 wt % of surface active agents; from about 0.6% wt % to about 1.2 wt % of film forming materials and from about 10 wt % to about 20 wt % of surface active agents; from about 1.2% wt % to about 2 wt % of film forming materials and from about 20 wt % to about 30 wt % of surface active agents; and from about 0.6% wt % to about 2 wt % of film forming materials and from about 3 wt % to about 10 wt % of surface active agents.

D. Suspended Benefit Agents

The personal care composition of the present invention may comprise a suspended benefit agent and. The suspended benefit agents comprise hydrophobic benefit materials, polymers, moisturizing agents, pigments, interference pigments, pearlescent agents, particles, exfoliating particles, shiny particles, beads, hydrophobically modified non-platelet particles, microcapsules, and mixtures thereof.

Additional suspended benefit agents, which can be used in the personal care compositions of the present invention, comprise microcapsules; thickening agents; low density microspheres (e.g. Expancel 091 WE40 d24, Akzo Nobel and others described in commonly owned and assigned U.S. Patent Publication No. 2004/0092415A1 published on May 13, 2004); polymeric phase structurant (e.g. naturally derived polymers, synthetic polymers, crosslinked polymers, block copolymers, copolymers, hydrophilic polymers, nonionic polymers, anionic polymers, hydrophobic polymers, hydrophobically modified polymers, associative polymers, and oligomers); a liquid crystalline phase inducing structurant (e.g. trihydroxystearin available from Rheox, Inc. under the trade name THIXCIN® R); organic cationic deposition polymer (e.g. Polyquatemium 10 available from Amerchol Corp. Edison, N.J., USA, guar hydroxypropyltrimonium chloride available as Jaguar C-17 from Rhodia Inc., and N-Hance polymer series commercially available from Aqualon); pigments; colorants; pearlescent agents; interference pigments (e.g such as those disclosed in U.S. Pat. No. 6,395,691 issued to Liang Sheng Tsaur, U.S. Pat. No. 6,645,511 issued to Aronson, et al., U.S. Pat. No. 6,759,376 issued to Zhang, et al, U.S. Pat. No. 6,780,826 issued to Zhang, et al.) particles (e.g. talc, kolin, mica, smectite clay, cellulose powder, polysiloxane, silicas, carbonates, titanium dioxide, polyethylene beads) hydrophobically modified non-platelet particles (e.g. hydrophobically modified titanium dioxide and other materials described in a commonly owned, patent application published on Aug. 17, 2006 under Publication No. 2006/0182699A by Taylor, et al.), polytetrafluoroethylene particles (e.g., PTFE particles available from MicroPowders, Inc. under the tradename Microslip). and mixtures thereof. The suspended benefit agents have an average particle size of about 1 μm about 1000 μm, in an alternate embodiment from about 2 μm to about 500 μm, and in an alternate embodiment about 5 μm to about 50 μm. In an alternate embodiment, the shaving composition contains from about 0% to about 60% by volume of suspended benefit agents having an average particle size of about 500 to about 1000 μm; from about 1 to about 50% by volume of suspended benefit agents having an average particle size of from about 40 μm to about 100 μm. The particles may be of any desired shape including spherical bead, elongated fiber or irregular shape, with spherical bead being the preferred shape. Generally the suspended benefit agents will be included in the shaving composition in an amount of about 0.01% to about 25%, in an alternate embodiment from about 0.1% to about 5%, by weight.

Without wishing to be bound by theory, it is believed that the addition of suspended benefit agents to shaving compositions imparts volume to the shaving composition, even when utilized with reduced or even no volatile lathering agents. Utilizing the shaving compositions of the present invention allows for an increase in amount of materials delivered to the skin, as suspended benefit agents are utilized to deliver volume instead of volatile lathering agents. This usage results in a reduced amount of gasses entrained within the composition, thus delivering more of the active agents in a given volume. These results are accomplished while still providing the volume and opacity that is preferred by the consumer.

E. Optional Components

Embodiments of the present invention may optionally contain a structure postponing agent (e.g., glycerin) that can slow the evaporation of the dominant solvent of the compositions, and therefore act to keep the compositions “wet” and lubricious during the total personal care (e.g., shaving) experience.

A non-limiting list of suitable structure postponing agents includes oils of all chain lengths and branchings, any cosmetically acceptable evaporation controllers, synthetic esters, and synthetic ethers, examples of which include: purcellin oil, isononyl isononanoate, isopropyl myristate, 2-ethylhexyl palmitate, 2-octyldodecyl stearate, 2-octyldodecyl erucate, isostearyl isostearate; hydroxylated esters such as isostearyl lactate, octyl hydroxystearate, octyldodecyl hydroxystearate, diisostearyl malate, triisocetyl citrate and fatty alkyl heptanoates, octanoates, decanoates, pentaerythritol esters, for instance pentaerythrityl tetraisostearate, derivatives of petroleum jelly, polydecenes, hydrogenated polyiosbutene both linear and branched, mineral oil, hydrocarbon-based oils of plant origin, liquid triglyceride like heptanoic or octanoic acid triglycerides or alternatively, for example, sunflower oil, corn oil, soybean oil, marrow oil, grapeseed oil, sesame oil, hazelnut oil, apricot oil, macadamia oil, arara oil, coriander oil, castor oil, avocado oil, caprylic/capric acid triglycerides. Emollients can also help control the rate of evaporation and the tackiness of the composition. Preferred emollients include mineral oil, lanolin oil, coconut oil, cocoa butter, olive oil, aloe extracts, jojoba oil, castor oil, fatty acids such as oleic and stearic acid, fatty alcohols such as cetyl and hexadecyl alcohol diisopropyl adipate, benzoic and hydroxybenzoic acid esters of C9-C15 alcohols, isononyl iso-nonanoate, volatile silicone oils, such as polymethylsiloxanes with a linear or cyclic silicone chain, and especially cyclomethicone silicone oils, for instance cyclopentasiloxane, cyclohexasiloxane and cyclotetrasiloxane, volatile or non-volatile polymethylsiloxanes (PDMSs) containing a linear or cyclic silicone chain such as cyclopolydimethylsiloxanes (cyclomethicones), for instance cyclohexadimethylsiloxane and cyclopentadimethylsiloxane polydimethylsiloxanes comprising alkyl, alkoxy or phenylsilicones, for instance phenyl trimethicones, phenyl dimethicones, phenyltrimethylsiloxydiphenylsiloxanes, diphenyl dimethicones, diphenylmethyldiphenyltrisiloxanes, 2-phenylethyltrimethyl siloxysilicates and polymethylphenylsiloxanes, and linear or branched, non-cyclic volatile silicone oils. Further examples include linear or branched monoalcohols containing from 1 to 8 carbon atoms, for instance ethanol, propanol, butanol, isopropanol and isobutanol; polyethylene glycols; polyols, for instance propylene glycol, polypropylene glycol, glycerol, isoprene glycol and butylene glycol; fatty alcohols like cetyl alcohol, stearyl alcohol, octyldodecanol, 2-butyloctanol, 2-hexyldecanol, 2-undecylpentadecanol, oleyl alcohol or linoleyl alcohol; polyol esters, for instance propylene glycol dioctanoate, neopentyl glycol diheptanoate and diethylene glycol diisononanoate, lower monoalcohols containing from 1 to 5 carbon atoms such as ethanol or isopropanol, glycols containing from 2 to 8 carbon atoms, such as propylene glycol, ethylene glycol, butylene glycol or dipropylene glycol, C3-C4 ketones and C2-C4 aldehydes, urea, citric acid, sorbitol, sugars, PEG-4.

Although not necessary to forming a useful personal care composition, other cosmetic ingredients may be advantageously added to improve the application aesthetics and/or achieve other benefits. For example, the composition may include one or more of the following components: beard wetting agents, skin conditioning agents (e.g., vitamins A, C and E, aloe, allantoin, panthenol, alpha-hydroxy acids, phospholipids, triglycerides, botanical oils, amino acids), foam boosters, emollients, humectants (e.g., glycerin, sorbitol, propylene glycol), fragrances, colorants, antioxidants, preservatives, and acute anti-irritants such as, for example, resorcin, bromelain, lanolin, resorcinol, PEG 7 olivate, zinc oxide, and acetylsalicylate.

F. Volatile Lathering Agents

Personal care compositions provided herein may optionally contain a volatile lathering agent that is mixed with the base compositions to enable the final personal care composition to foam when dispensed from an appropriate container and/or when manipulated by a consumer with their hand or an applicator. Exemplary lathering agents include saturated aliphatic hydrocarbons having 4 to 6 carbon atoms, such as n pentane, isopentane, neopentane, n butane, isobutane, and mixtures thereof. The lathering agent may be selected so as to provide a vapor pressure at 20° C. of about 3 to about 20 psig, preferably about 5 to about 15 psig. Other lathering agents known to the skilled artisan may also be employed.

### II. Product Form and Application

Further, personal care compositions of the present invention may take on a variety of product forms including solids, creams, brushable gels and creams, non-foaming liquids, mechanically pumpable liquids, non-aerosol gels, aerosol gels, aerosol foams, pastes, serums, powders, strips, sprays, and sticks. The product form can impact the feasibility and degree to which new ingredient technology is compatible and noticeable to the consumer, as well as the effect on mess, speed of use, degree/level of portability and disposability, and effect on visual aesthetics. For example, higher polymer to surface active agent ratios may be achievable in a non-aerosol cream or gel because the packaging associated with the same is likely to present less shear and stringiness to the formulation during dispensing relative to high shear delivery vehicles typically associated with aerosol products.

### III. Shaving Products, Kits, Systems, and Methods

A variety of shaving products are provided by the present invention. One exemplary shaving product comprises a package, a personal care composition as described herein disposed within the package, and a propellant disposed within the package for facilitating dispensing of the personal care composition.

Another exemplary shaving product comprises a package, a personal care composition as described herein disposed within the package, and an applicator associated with the package for applying and lathering the personal care composition. The applicator can, for example, comprise an applicator head that is integrally affixed to the package such that the package itself serves as a handle, and may contain natural and/or synthetic fibers, a foam or sponge, an apertured film, a woven or nonwoven substrate, or mixtures thereof. The applicator may be bundled with the package whereby a user must affix it to the package. The applicator may also be bundled with the package but used separately from the package rather than using the package itself as a handle. Exemplary applicators that may be suitable for embodiments of the present invention include those shown and described in the following patent documents: U.S. Pat. No. 6,003,523; U.S. Pat. No. 4,636,102; U.S. Pat. No. 4,603,992; U.S. Pat. No. 4,963,047; U.S. Pat. No. 4,361,228; U.S. Pat. No. 4,348,126; and U.S. Pat. No. 4,252,455.

Shaving kits are also provided comprising a razor or razor blade cartridge, and a personal care composition as described herein, in liquid or dried down form, associated with the razor or blade cartridge. For example, the personal care compositions provided herein could be formulated into a solid or soft solid form and be provided in proximity to the blade such that the composition is applied to the skin as the blade is passed over the skin. Also, a personal care composition in liquid form could be dispensed from a reservoir or container that is associated with the razor or razor blade cartridge.

Methods of shaving are also provided, comprising applying a volume of a personal care composition described herein to an area of skin to be shaved, generating a lather with the personal care composition before, during, and/or after completing the application step, and shaving the area of skin to be shaved. Any residual composition remaining on the skin can either be rinsed off or rubbed into the skin after shaving.

### IV. Examples

The following examples further describe and demonstrate embodiments within the scope of the present invention. The examples are given solely for the purpose of illustration and are not to be construed to limit scope of the present invention as many variations thereof are possible without departing from the spirit and scope of the invention.

| | Ex. 1 | Ex. 2 | Ex. 3 | Ex. 4 | Ex. 5 | Ex. 6 |
|---|---|---|---|---|---|---|
| Ingredient | Base | Base | Base | Base | Base | Base |
| Water | QS | QS | QS | QS | QS | QS |
| Palmitic Acid | 7.75 | 6.0 | 0 | 0 | 6.0 | 0 |
| Triethanolamine | 6.05 | 4.7 | 8.76 | 8.76 | 4.7 | 8.76 |
| Stearic Acid | 2.6 | 2.0 | 0 | 0 | 2 | 0 |
| Myristic Acid | 0 | 0 | 14.0 | 14.0 | 0 | 14.0 |
| Mineral Oil kaydol | 0 | 0 | 0 | 0 | 1.5 | 0 |
| Glyceryl Oleate | 2.0 | 2.0 | 1.5 | 1.5 | 2.5 | 1.5 |
| Sorbitol (70% solution) | 1.0 | 1.0 | 0 | 0 | 0.5 | 0 |
| Hydroxyethylcellulose | 0.5 | 0.3 | 0.75 | 0.75 | 0.3 | 0.75 |
| Polyox WSR-301 | 0.06 | 0.06 | 0 | 0 | 0.06 | 0 |
| (PEG-90M) | | | | | | |
| Polyox WSR-N-12K | 0.05 | 0.05 | 0 | 0 | 0.05 | 0 |
| (PEG-23M) | | | | | | |
| Sodium Myristoyl | 1.0 | 0 | 6.67 | 6.67 | 0 | 6.67 |
| Sarcosinate (30%) | | | | | | |
| Polymer LR30M cationic | 0.1 | 0.2 | 0.45 | 0.45 | 0.2 | 0.45 |
| Fragrance | 0.85 | 0.85 | 0.85 | 0.85 | 0.85 | 0.85 |
| PVP K60 (45% solution) | 0 | 0 | 0.49 | 0.49 | 0 | 0.49 |
| Glycerin | 0.5 | 1.5 | 8.5 | 8.5 | 0.5 | 8.5 |
| Colorant | 0.002 | 0.002 | 0.002 | 0.002 | 0.002 | 0.002 |
| Parts of Base Mixed w/Lathering | 97.15 | 97.15 | 96 | 97.15 | 97.15 | 100 |
| Agent | | | | | | |
| Volatile Lathering Agent | 2.85 | 2.85 | 4.0 | 2.85 | 2.85 | 0 |

Examples 1-6 are shaving compositions with varying levels of a film forming system and surface active agent. These examples can be made as follows: Mix the water with glycerin and then add to this mixture a pre-blend of the LR30M and hydroxyethylcellulose powders. For formulas containing sodium myristoyl sacrosinate, add this component next. For formulas containing PVP K60, add this component next. Mix until homogeneous and start heating. Stop mixing, and then add the fatty acids. When the temperature reaches 75° C., add triethanolamine and mix for approximately 10 more minutes. Cool to less than 35° C., and then add the fragrance and colorant. For example 6, store in closed container until it is charged into an appropriate dispenser. For examples 1-5, combine the base composition with the lathering agent and charge the mixture into containers or packages capable of containing volatile agents.

| Ingredient | Ex. 7 | Ex. 8 | Ex. 9 | Ex. 10 | Ex. 11 | Ex. 12 |
|---|---|---|---|---|---|---|
| Water | QS | QS | QS | QS | QS | QS |
| Palmitic Acid | 0 | 6.0 | 10.0 | 4.0 | 1.0 | 0 |
| Triethanolamine | 0 | 18.0 | 0 | 12.0 | 9.0 | 0 |
| Stearic Acid | 0 | 6.0 | 14.0 | 4.0 | 0 | 0 |
| Myristic Acid | 0 | 5.0 | 10.0 | 5.0 | 8.0 | 0 |
| Mineral Oil kaydol | 0 | 2.0 | 0 | 0 | 0 | 0 |
| Glyceryl Oleate | 0 | 0 | 0 | 1.0 | 0 | 0 |
| Carbopol Ultrez 10 | 0.7 | 0 | 0 | 0 | 0 | 0 |
| Hydroxyethylcellulose | 0.25 | 1.0 | 0.3 | 1.0 | 1.0 | 0.35 |
| Polypropylene Glycol | 3.0 | 3.0 | 0 | 0 | 0 | 3.0 |
| Sodium Hydroxide (50% solution) | 0.74 | 0 | 20.0 | 0 | 0 | 0 |
| Sodium Myristoyl Sarcosinate (30%) | 10.0 | 0 | 0 | 4.0 | 8.0 | 9.7 |
| Polymer LR30M cationic | 0.65 | 0.2 | 0 | 0.35 | 0.35 | 0.6 |
| Polyethylene Oxide | 0 | 0 | 0 | 0 | 0 | 0.25 |
| Fragrance | 0.75 | 1.55 | 1.0 | 1.0 | 1.0 | 0.75 |
| PVP K80 (100% powder) | 0.25 | 2.0 | 0.3 | 0.24 | 0.3 | 0 |
| Glycerin | 10.0 | 15.0 | 7.0 | 9.0 | 3.0 | 10.0 |
| Colorant | 0.003 | 0 | 0 | 0.003 | 0 | 0.003 |
| Olive Oil | 0 | 0 | 17.25 | 0 | 0 | 0 |
| Polyhydroxystearic acid/Isononyl | 0 | 0 | 0 | 0 | 11.2 | 0 |
| Isononoate/Ethylhexyl | | | | | | |
| Isononanoate/Sodium | | | | | | |
| Cocamidopropyl PG-Dimonium | | | | | | |
| Chloride Phosphate | | | | | | |
| Laponite Clay | 0 | 0 | 0 | 0 | 0 | 1.2 |

| Ingredient | Ex. 13 | Ex. 14 | Ex. 15 | Ex. 16 | Ex. 17 |
|---|---|---|---|---|---|
| Water | QS | QS | QS | QS | QS |
| Palmitic Acid | 0 | 2.0 | 0 | 2.0 | 3.0 |
| Triethanolamine | 11.0 | 12.0 | 9.35 | 9.0 | 9.0 |
| Stearic Acid | 0 | 2.0 | 0 | 2.0 | 3.0 |
| Myristic Acid | 13.35 | 9.0 | 14.0 | 9.0 | 3.0 |
| Glyceryl Oleate | 1.0 | 0 | 0 | 0 | 0 |
| Hydroxyethylcellulose | 0.75 | 1.0 | 0.75 | 1.0 | 0.4 |
| Sodium Myristoyl | 6.0 | 7.3 | 6.0 | 5.45 | 4.0 |
| Sarcosinate (30%) | | | | | |
| Polymer LR30M cationic | 0.45 | 0.4 | 0.45 | 0 | 0.3 |
| Fragrance | 1.0 | 1.0 | 1.0 | 1.0 | 1.0 |
| PVP K80 (100% powder) | 0.3. | 0.3 | 0.3 | 0.4 | 0.3 |
| Ethanol | 0 | 0 | 0 | 0 | 1.0 |
| Glycerin | 5 | 8.5 | 10.0 | 0 | 0 |
| Colorant | 0.003 | 0.004 | 0.003 | 0.003 | 0 |
| Isopentane | 4.0 | 0 | 0 | 0 | 3.5 |
| Perfluorobutyl ether, | 0 | 0 | 0 | 12.0 | 0 |
| methyl perfluorobutyl | | | | | |
| ether, Polyhdroxystearic | | | | | |
| acid/Isononyl | | | | | |
| Isononanoate/ | | | | | |
| Ethylhexyl Isononanoate/ | | | | | |
| Sodium Cocamidopropyl | | | | | |
| PG-Dimonium Chloride | | | | | |
| Phosphate | | | | | |
| Polyhydroxystearic acid/ | 0 | 0 | 0 | 0 | 10.0 |
| Isononyl Isononoate/ | | | | | |
| Ethylhexyl Isononanoate/ | | | | | |
| Sodium Cocamidopropyl | | | | | |
| PG-Dimonium Chloride | | | | | |
| Phosphate | | | | | |
| Copolymer of vinylidene | 0 | 0.35 | 0 | 0 | 0 |
| chloride/acrylonitrile/ | | | | | |
| methylmethacrylate | | | | | |

| | Ex. 18 | Ex. 19 |
|---|---|---|
| Ingredient | Base | Base |
| Water | QS | QS |
| Palmitic Acid | 9.0 | 5.9 |
| Triethanolamine | 9.0 | 9.5 |
| Stearic Acid | 10.0 | 2.5 |
| Myristic Acid | 0 | 9.5 |
| Coconut Oil | 5.0 | 0 |
| Glyceryl Oleate | 1.66 | 0 |
| Sorbitol (70% solution) | 0.827 | 0 |
| Natrosol 250HHR | 0.414 | 1.15 |
| Polyox WSR-301 (PEG-90M) | .05 | 0 |
| Polyox WSR-N-12K (PEG-23M) | .0410 | 0 |
| Polymer LR-30M Cationic | 0 | 0.15 |
| Luviskol K-80 | 0 | 0.24 |
| Crodasinic MS-30 | 0 | 8.0 |
| Glydant Plus (Preservative) | 0.25 | 0.25 |
| Fragrance | 1.3 | 1.0 |
| Micro Slip 519 (PTFE) | 0.25 | 0 |
| Glycerin | 2.5 | 5.0 |
| Expancel 920 | 0.25 | 0 |
| Potassium Hydroxide solution (50%) | 3.5 | 0 |
| Acusol-OP301 | 0 | 0.25 |

Examples 7-19 can be made as follows: Separately pre-blend the colorant with 5% reserved water from original batch. Mix the remaining water and with the other polar liquids (e.g., glycerin). Blend the polymer raw materials in powder form and add to the water/polar liquid mixture. Add surface active agents that can improve wetout and mixing of the polymers. Mix until homogeneous and start heating. Stop mixing, and then add the fatty acids. When the temperature reaches 75° C., add triethanolamine and mix for approximately 10 more minutes. After cooling add Micro Slip, Expancel, or Acusol as required. Cool to less than 35° C., and then add the fragrance and colorant pre-blend.

### V. Performance

Table 1 illustrates comparative data among commercially available shaving compositions, including aerosol gel, aerosol foam, and non-aerosol products, and Examples 18 and 19 from above. Viscosity measurements were made as described herein. Friction Coefficients were determined by applying 50 grams of the compositions on a polyurethane artificial skin, and dragging a fixed weight across the artificial skin and measuring using a Diastron Friction Tester. (Typically friction near 1.0 means a rough surface, and friction 0-0.7 means fairly slippery smooth surface.) The Protection Factor is determined by in dividing the viscosity by the friction coefficient. The example compositions have surprisingly high protection factors as compared to the commercially available shaving compositions, while having a friction coefficient within 0.055 of the average of this group.

TABLE 1

| | Viscosity | Friction | Protec- |
|---|---|---|---|
| | 1000⁻¹ | Coeffi- | tion |
| Shave Composition | cps | cient | Factor |
| Edge Sensitive Skin -Aerosol Gel | 19.4 | 0.347 | 55.9 |
| Noxema for Sensitive Skin - Aerosol | 25.3 | 0.375 | 67.5 |
| Foam | | | |
| Barbasol Original - Aerosol Foam | 29.6 | 0.389 | 85.1 |
| Nivea for Men Sensitive - Aerosol Gel | 7.9 | 0.359 | 22.0 |
| Neutrogena Skin Clearing Shave Cream - | 176.4 | 0.299 | 590.0 |
| Nonaerosol | | | |
| King of Shaves - Alphagel Nonaerosol | 87.1 | 0.327 | 266.4 |
| Example 18 | 1750.9 | 0.400 | 4333.9 |
| Example 19 | 263.3 | 0.375 | 702.1 |

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description section are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

It should be understood that every maximum numerical limitation given throughout this specification will include every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1) A shaving composition, comprising:
(a) a base composition, comprising:
i) greater than or equal to about 0.6%, by weight of the base composition, of a film forming system comprising one or more film forming materials; and
ii) one or more water soluble or water dispersible surface active agents, wherein the ratio of the one or more film forming materials to the one or more surface active agents is from about 0.03 to about 0.7; and
(b) at least one suspended benefit agent disposed within the base composition.

2) The shaving composition of claim 1, wherein the base composition comprises greater than or equal to about 1.0% of the one or more film forming materials.

3) The shaving composition of claim 1, wherein the one or more film forming materials comprises a cationic polymer.

4) The shaving composition of claim 3, wherein the cationic polymer comprises a polyquaternium.

5) The shaving composition of claim 1, wherein the one or more film forming polymers comprises both a cationic polymer and a lubricant polymer having a molecular weight of greater than about 150,000 g/mol.

6) The shaving composition of claim 1, wherein the one or more film forming polymers is selected from the group consisting of a water soluble cellulose ether, a polyethylene oxide, polyvinylpyrrolidone, a polyquaternium, and mixtures thereof.

7) The shaving composition of claim 1, wherein the ratio of the one or more film forming polymers to the one or more surface active agents is from about 0.035 to about 0.35.

8) The shaving composition of claim 1, wherein the base composition comprises less than about 30% of the one or more surface active agents.

9) The shaving composition of claim 1, wherein the base composition comprises less than about 20% of the one or more surface active agents.

10) The shaving composition of claim 1, wherein the base composition comprises less than about 10% of the one or more surface active agents.

11) The shaving composition of claim 1, wherein the base composition comprises less than about 5% of the one or more surface active agents.

12) The shaving composition of claim 1, wherein the shaving composition comprises less than about 10% by weight of water.

13) The shaving composition of claim 1, wherein the shaving composition, wherein the shaving composition comprises a viscosity of from about 185 to about 2,500 cps.

14) The shaving composition of claim 1, wherein the shaving composition comprises from about 0.01% to about 25%, of the suspended benefit agent.

15) A shaving composition, comprising:
(a) a base composition, comprising:
i) greater than about 50%, by weight of the base composition, of water;
ii) from about 3% to about 30%, by weight of the base composition, of one or more water soluble or water dispersible surface active agents; and
iii) from about 0.6% to about 2.0%, by weight of the base composition, of one or more film forming materials; and
(b) from about 0.01% to about 25% of at least one suspended benefit agent disposed within the base composition
(c) wherein the shaving composition comprises a viscosity of from about 185 to about 2,500 cps.

16) The shaving composition of claim 15, wherein the base composition comprises from about 20% to about 30% of the one or more surface active agents, and from about 0.6% to about 1.2% of the one or more film forming materials.

17) The shaving composition of claim 15, wherein the base composition comprises from about 10% to about 20% of the one or more surface active agents, and from about 0.6% to about 1.2% of the one or more film forming materials.

18) The shaving composition of claim 15, wherein the base composition comprises from about 20% to about 30% of the one or more surface active agents, and from about 1.2% to about 2.0% of the one or more film forming materials.

19) The shaving composition of claim 15, wherein the base composition comprises from about 3% to about 10% of the one or more surface active agents, and from about 0.6% to about 1.2% of one or more film forming materials.

20) A shaving composition, comprising:
(a) a base composition, comprising:
i) greater than about 50%, by weight of the base composition, of water;
ii) from about 3% to about 30%, by weight of the base composition, of one or more water soluble or water dispersible surface active agents; and
iii) from about 0.6% to about 2.0%, by weight of the base composition, of one or more film forming materials; and
(b) from about 0.1% to about 5%, of at least one suspended benefit agent disposed within the base composition
(c) wherein the shaving composition comprises a viscosity of from about 185 to about 2,500 cps.
